(19) United States
(12) Patent Application Publication
(10) Pub. No.: US 2014/0023585 A1
(43) Pub. Date: Jan. 23, 2014

(54) 177 LUTETIUM-LABELED BOMBESIN ANALOGS FOR RADIOTHERAPY

(71) Applicants: Sandra Borkowski, Hohen Neuendorf (DE); Rosalba Mansi, Munchenstein (CH); Helmut Macke, Lorrach (DE)

(72) Inventors: Sandra Borkowski, Hohen Neuendorf (DE); Rosalba Mansi, Munchenstein (CH); Helmut Macke, Lorrach (DE)

(73) Assignees: UNIVERSITATSSPITAL BASEL, BASEL (CH); PIRAMAL IMAGING SA, MATRAN (CH)

(21) Appl. No.: 13/988,629

(22) PCT Filed: Nov. 21, 2011
(86) PCT No.: PCT/EP2011/070553

§ 371 Date: Oct. 7, 2013

(30) Foreign Application Priority Data
Nov. 22, 2010 (EP) 10075743.4

(51) Int. Cl. A61K 51/08

(52) U.S. Cl. CPC A61K 51/088
USPC 424/1.69; 530/328

(57) ABSTRACT

The invention is directed to novel Lutetium-177-labeled bombesin analogs for treatment of tumor by radiotherapy.

### FIELD OF INVENTION

The invention is directed to novel Lutetium-177-labeled bombesin analogs for treatment of tumor by radiotherapy.

### BACKGROUND ART

Radiation therapy is the most common modality of cancer treatment; across the world annually 50% of the cancer patients receive radiation administration. Generally beams of particles are used to treat malignant tissue, using photon (x-ray /γ-ray), or electron, which produce low linear-energy transfer to the tissue. These beams are generated usually by means of linear accelerators or radioactive sources. These types of radiotherapy or radiosurgery facilities are widely used in clinics and hospitals. However, the main problem is that, in conventional radiation therapy, it is difficult to eradicate the cancer cells successfully and tumour recurrence occurs which causes therapeutic failure. Furthermore, normal tissue is also affected considerably, producing radiation toxicity. Side-effects such as inflammation of the radiated site are common, and in the brain there can be toxic necrosis and gliosis, together with associated dementia and cognitive deterioration which is a serious side-effect of radiotherapy in neuro-oncology. The successful treatment of cancer with radiotherapy requires a large radiation dose for accumulating accurately at tumor position and specifically targeting tumors. Molecular targeted radiotherapy is therefore thought to be a promising approach to fullfill this aim of effective and selective tumor dosage combined with reduced side-effects to healthy tissue.

Peptides are biomolecules that play a crucial role in many physiological processes including actions as neurotransmitters, hormones, and antibiotics. Research has shown their importance in such fields as neuroscience, immunology, pharmacology and cell biology. They bind to receptor on the target cell surface and the biological effect of the ligand is transmitted to the target tissue. Tumors overexpress various receptors types to which peptides can bind specifically. Boerman et al. (Seminar in Nuclear Medicine, 30(3) July, 2000; pp 195-208) and Schottelius et al. (Methods, 48(2), June 2009; 161-177) provide a non exhaustive, but comprehensive list of peptides binding to receptor involved in tumor, i.e., somatostatin, vasoactive intestinal peptide (VIP), bombesin binding to gastrin-releasing peptide (GRP) receptor, gastrin, cholecystokinin (CCK) and calcitonin.

The Bombesin peptide has been shown to be overexpressed in BB2 receptors in prostate cancer. Radiopeptide therapy is well known to be effective in the case of neuroendocrine tumors using radiolabeled (Y-90. Lu-177, or In-111) somatostatin analogs (Bodei L. et al. Eur Rev Med Pharmacol Sci. 2010 April; 14(4):347-51). Also bombesin analogs targeting the gastrin-releasing-peptide receptor (GRPr), were aimed for radiopeptide therapy of human tumors with Lu-177-AMBA as the most prominent example in clinical development (Lantry L E et al., J Nucl Med. 2006 July; 47(7):1144-52). However, the most critical organ using these radiolabeled peptides are the kidneys being sensitive to radiation. Elevated kidney uptake and retention potentially produces severe side effects (e.g. nausea) and acute or chronic nephrotoxicity. Somatostatin-based radiopeptide therapy is therefore adapted to a dose-regimen preventing especially kidney toxicity and also hematotoxicity as the next critical side-effect.

CB-TE2A is a cross-bridged monoamides that is a stable chelation system for ^{64/67}Cu that was incorporated with Bombesin analogs for in vitro and in vivo studies of prostate cancer. PET/CT imaging studies showed that it underwent uptake into prostate tumor xenographs selectively with decreased uptake into non target tissues, Parry, Jesse J. “MicroPET imaging of breast cancer using radiolabeled bombesin analogs targeting the gastrin-releasing peptide receptor.” Springer 101 (2007): 175-183.

Theoretically, the high affinity of the ligand for the receptor, the pharmacokinetics of the ligand and the accessability of the antigen facilitate retention of the radiolabeled ligand in receptor expressing tissues and its clearance from non-target organs which may be altered during chemical reaction. Therefore an optimal peptidic construct has to be designed. A key moiety is the linkage of the radionuclide to the biomolecule. Various methods have been described resulting in the presence or absence of a linker between the radionuclide and the biomolecule. Hence, various linkers are known. For example, C. J. Smith at al. (Nucl. Med. Bio., 30(2):101-9; 2003) disclose radiolabeled bombesin wherein the linker is DOTA-X where X is a ω-NH₂—(CH₂)₇—COOH (8-Aoc).

The object of the present invention is to provide improved radiotherapeutic agents based on bombesin peptide antagonists which have been shown their potential as imaging agents for effective radiopeptide therapy of human GRPr expressing tumors.

### SUMMARY OF THE INVENTION

The object of the present invention is solved in detail herein below. The present invention is directed to compounds of Formula I, to a method for obtaining compounds of Formula I and method for treatment of tumor by radionuclide therapy (radiotherapy).

### DRAWINGS

FIG. 1: Binding affinity of compound 2 [^{177/nat}Lu] and compound 3 [^{111/nat}In].

FIG. 2: Serum Stability of compound 2 [^{177/nat}Lu].

FIG. 3: Dosimetry of compound 2 in PC-3-bearing mice.

FIG. 4: Radionuclide therapy Study of 100 pmol/6 MBq of compound 2.

FIG. 5: Radionuclide therapy Study of 200 pmol/12 MBq of compound 2.

FIG. 6: Radionuclide therapy Study of 400 pmol/24 MBq of compound 2.

FIG. 7: Radionuclide therapy Study of 200 pmol of ^{nat}Lu-compound 2.

FIG. 8: Radionuclide therapy Study of control with PBS (100 μL).

FIG. 9: Radionuclide therapy Study of 37 MBq of compound 2 with Single injection.

FIG. 10: Radionuclide therapy Study of 37 MBq of compound 2 with Single injection.

### DETAILED DESCRIPTION OF THE INVENTION

In a first aspect, the present invention is directed to bombesin analog peptide antagonist compounds or conjugates of formula I

[¹⁷⁷Lu]—R¹—R²—R³ (I)

wherein
R¹ metal chelator suitable for chelating [¹⁷⁷Lu],
R² spacer linked to N-terminal of R³ or a covalent bond,
R³ bombesin analog peptide antagonist of sequence from seq 1 to 4
Seq 1: D-Phe-Gln-Trp-Ala-Val-Gly-His-Sta-Leu-NH₂;
Seq 2: D-Phe-Gln-Trp-Ala-Val-Gly-His-Leuψ(CHOH—CH₂)—(CH₂)₂—CH₃;
Seq 3: D-Phe-Gln-Trp-Ala-Val-Gly-His-Leuψ(CH₂NH)-Phe-NH₂, and
Seq 4: D-Phe-Gln-Trp-Ala-Val-Gly-His-Leuψ(CH₂NH)-Cys-NH₂.
and pharmaceutically acceptable salt.

ψ indicates that the amide carbonyl (C═O) is replaced with CH₂
For example:

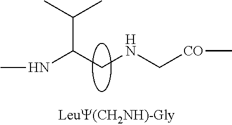

The invention further refers to suitable salts of inorganic or organic acids, and hydrates of the compounds of Formula I.

Preferably, the metal chelator R¹ suitable for chelating [¹⁷⁷Lu] is selected from the group comprising:
DOTA-, NODASA-, NODAGA-, NOTA-, DTPA-, EDTA-, TETA-, and TRITA-based chelators and their close analogs.

DOTA stands for 1,4,7,10-tetrazacyclododecane-N, N′,N″,N′″ tetraacetic acid.

DTPA stands for diethylenetriaminepentaacetic acid.

EDTA stands for ethylenediamine-N,N′-tetraacetic acid.

TETA stands for 1,4,8,11-tetraazacyclododecane-1,4,8,11-tetraacetic acid.

NOTA stands for 1,4,7-triazacyclononane-1,4,7-triacetic acid.

NODASA stands for 1,4,7-TRIAZACYCLONONANE-1-SUCCINIC ACID-4,7-DIACETIC ACID.

NODAGA stands for 1,4,7-triazacyclononane-N-glutaric acid-N′,N″-diacetic acid.

TRITA stands for 1,4,7,10 tetraazacyclotridecane-1,4,7,10 N,N′,N″,N′″-tetraacetic acid.

More preferably, the metal chelator R¹ is selected from the group comprising:
DOTA-, NOTA-, DTPA-, and TETA-based chelators.

The structures of these chelating ligands in their fully deprotonated form are shown below.

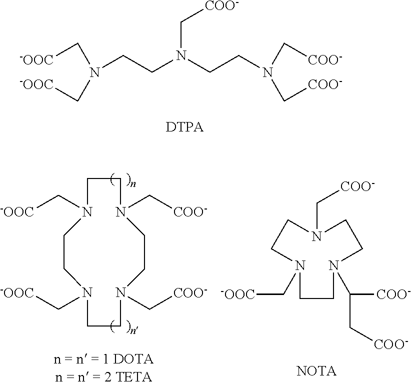

Even more preferably, the metal chelator R¹ is DOTA (1,4,7,10-tetrazacyclododecane-N, N′,N″,N′″ tetraacetic acid).

Preferably, R² is a spacer linked to N-terminal of R³ having formula II

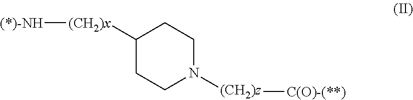

wherein
x is an integer from 0 to 3,
z is an integer from 0 to 3;
(*) linked to R¹ and
(**) linked to R³.

More preferably,
x is 0, and
z is 1 (CH₂);
x/z=1 means CH₂.
x/z=2 means CH₂—CH₂.
x/z=3 means CH₂—CH₂—CH₂.

Preferably, R³ is
Seq 1: D-Phe-Gln-Trp-Ala-Val-Gly-His-Sta-Leu-NH₂.

Additionally, the functional sites of the bombesin peptide R³ are protected by employing groups for blocking or protecting the functional sites such as carboxylic acid or amine moieties. The invention conjugate of formula (I) is optionally a protected conjugate wherein the functional site(s) of bombesin peptide is protected Preferably. Seq 1 is protected Gln(Trt)-Trp(Boc)-Ala-Val-Gly-His(Trt)-Sta-Leu-NH-(Seq 1 protected wherein protecting groups are triphenyl-methyl (trt) or tert-butyloxycarbonyl (Boc).

O-protecting group is selected from the group comprising
Methyl, Ethyl, Propyl, Butyl and t-Butyl. Preferably, O-protecting group is selected from the group comprising Methyl. Ethyl and t-Butyl. More preferably, O-protecting group is t-Butyl. N-protecting group is selected from the group comprising
Carbobenzyloxy (Cbz), tert-Butyloxycarbonyl (BOC), 9-Fluorenylmethyloxycarbonyl (FMOC), and Triphenylmethyl. Preferably, N-protecting group is selected from the group comprising Carbobenzyloxy (Cbz), tert-Butyloxycarbonyl (BOC) and 9-Fluorenylmethyloxycarbonyl (FMOC). More preferably, N-protecting group is tert-Butyloxycarbonyl (BOC) or 9-Fluorenylmethyloxycarbonyl (FMOC).

Preferred compound of formula I is

| Radioisotope | R¹ Chelator | R² Spacer | R³ Bombesin sequence |
|---|---|---|---|
| [¹⁷⁷Lu] | DOTA- | 4-amino-1-carboxymethyl-piperidine- | D-Phe-Gln-Trp-Ala-Val-Gly-His-Sta-Leu-NH2 |

[¹⁷⁷Lu]-DOTA-4-amino-1-carboxymethylpiperidine-D-Phe-Gln-Trp-Ala-Val-Gly-His-Sta-Leu-NH₂

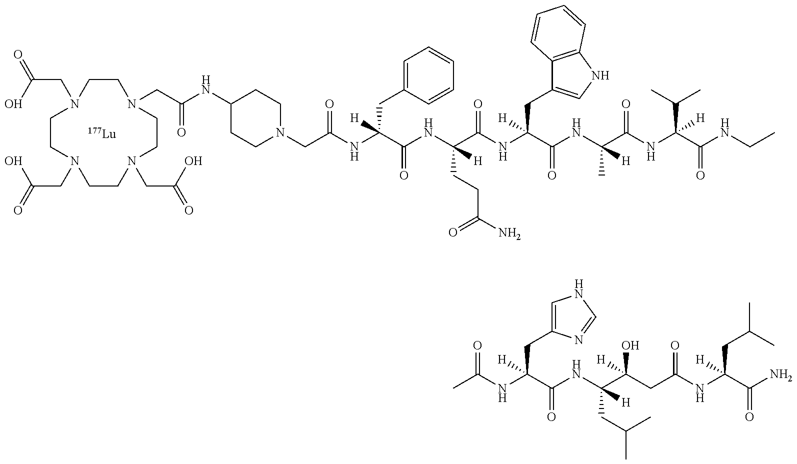

In a second aspect, the present invention is directed to composition comprising a compound of Formula I and pharmaceutically acceptable carrier or diluent. The person skilled in the art is familiar with auxiliaries, vehicles, excipients, diluents, carriers or adjuvants which are suitable for the desired pharmaceutical formulations, preparations or compositions on account of his/her expert knowledge.

The administration of the compounds, pharmaceutical compositions or combinations according to the invention is performed in any of the generally accepted modes of administration available in the art. Intravenous deliveries are preferred.

Preferably the composition comprises [¹⁷⁷Lu]-DOTA-4-amino-1-carboxymethylpiperidine-D-Phe-Gln-Trp-Ala-Val-Gly-His-Sta-Leu-NH₂ and pharmaceutically acceptable carrier or diluent

In a third aspect, the present invention is directed to a method for radiotherapy of a cancer patient using the compound of formula I as radiotherapeutic agent.

The patient is any mammal such as an animal or a human, preferably a human.

The radiotherapeutic agent is a compound of formula I and preferably, is [¹⁷⁷Lu]-DOTA-4-amino-1-carboxymethylpiperidine-D-Phe-Gln-Trp-Ala-Val-Gly-His-Sta-Leu-NH₂. A cancer patient is a patient that was diagnosed with a proliferative diseases wherein proliferative diseases are cancer characterised by the presence of tumor and/or metastases. Preferably, tumor and/or metastases are located in or originated from the prostate, lung or breast.

The invention relates also to a conjugate/compound of formula I or a pharmaceutical composition thereof for radiotherapy of cancer.

The invention relates also to the use of a compound of formula I or a pharmaceutical composition thereof for the manufacture of a radiotherapeutic agent for treatment of cancer.

The method for radiotherapy comprises the steps of administering to a subject in need thereof compound of formula I or composition thereof in therapeutically effective amounts, and after localization of compound of formula I or composition in the desired tissues, subjecting the tissues to irradiation to achieve the desired therapeutic effect.

The compounds of this invention are useful for the imaging of a variety of cancers wherein the receptor Gastrin Releasing Peptid (GRP) is over expressed.

Preferably, cancer includes but not limited to: carcinoma such as bladder, breast, colon, kidney, liver, lung, including small cell lung cancer, esophagus, gall-bladder, ovary, pancreas, stomach, cervix, thyroid, prostate and skin, hematopoetic tumors of lymphoid and myeloid lineage, tumors of mesenchymal origin, tumors of central peripheral nervous systems, other tumors, including melanoma, seminoma, teratocarcinoma, osteosarcoma, xeroderma pigmentosum, keratoxanthoma, thyroid follicular cancer and Karposi's sarcoma.

Preferably, the present invention will be useful for imaging prostate cancer; lung or breast cancer and resulting tumor thereof, more preferably prostate cancer.

The radioactively labeled compounds according to Formula I provided by the invention may be administered intravenously in any pharmaceutically acceptable carrier, e.g., conventional medium such as an aqueous saline medium, or in blood plasma medium, as a pharmaceutical composition for intravenous injection. Such medium may also contain conventional pharmaceutical materials such as, for example, pharmaceutically acceptable salts to adjust the osmotic pressure, buffers, preservatives and the like. Among the preferred media are normal saline and plasma. Suitable pharmaceutical acceptable carriers are known to the person skilled in the art. In this regard reference can be made to e.g., Remington's Practice of Pharmacy, 11^{th} ed. and in J. of. Pharmaceutical Science & Technology, Vol. 52, No. 5, September-October, p. 238-311 see table page 240 to 311, both publication include herein by reference.

The concentration of the compound of Formula I and the pharmaceutically acceptable carrier, for example, in an aqueous medium, varies with the particular field of use. A sufficient amount is present in the pharmaceutically acceptable carrier when satisfactory visualization of the imaging target (e.g., a tumor) is achievable.

In accordance with the invention, the radiolabeled compounds of Formula I either as a neutral composition or as a salt with a suitable counter-ion are administered in a single unit injectable dose. Any of the common carriers known to those with skill in the art, such as sterile saline solution or plasma, can be utilized after radiolabelling for preparing the injectable solution in accordance with the invention. In comparison to somatostatin-based radiopeptide therapy, the unit dose to be administered for a radiotherapy agent depending on radiosensitive dose-critical organs (usually about 4-8 GBq per cycle; 3 cycles) is increased with the invented bombesin antagonists of Formula I to about 1-50 GBq.

In a fourth aspect, the present invention is directed to a method for obtaining a bombesin analog peptide antagonist conjugate of formula I

[¹⁷⁷Lu]—R¹—R²—R³ (I)

wherein
R¹ metal chelator suitable for chelating [¹⁷⁷Lu],
R² spacer linked to N-terminal of R³ or a covalent bond,
R³ bombesin analog peptide antagonist of sequence from seq 1 to 4
Seq 1: D-Phe-Gln-Trp-Ala-Val-Gly-His-Sta-Leu-NH₂;
Seq 2: D-Phe-Gln-Trp-Ala-Val-Gly-His-Leuψ(CHOH—CH₂)—(CH₂)₂—CH₃;
Seq 3: D-Phe-Gln-Trp-Ala-Val-Gly-His-Leuψ(CH₂NH)-Phe-NH₂, and
Seq 4: D-Phe-Gln-Trp-Ala-Val-Gly-His-Leuψ(CH₂NH)-Cys-NH₂.
   comprising the step
   Optionally coupling a spacer R² to a bombesin analog peptide antagonist R³ for obtaining R²—R³ (step 1),
   Coupling R²—R³ to a suitable chelator (step 2) and
   Radiochelating the bombesin analog peptide antagonist conjugate R¹—R²—R³ with [¹⁷⁷Lu] (step 3).

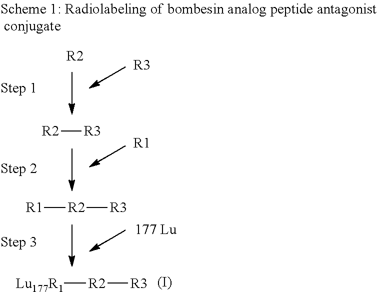

Preferably, the method for preparing a bombesin analog peptide antagonist conjugate having general Formula (I) comprises the step of radiochelating with [¹⁷⁷Lu] (step 3).

R¹, R² and R³ are defined as above.

The obtained compound is optionally deprotected at the protected functional site(s).

Embodiments and preferred features can be combined together and are within the scope of the invention. The preferred features disclosed for compound of general formula (I) are incorporated herein.

In a fifth aspect, the present invention is directed to a kit comprising a sealed vial containing a predetermined quantity of a compound having general chemical Formula (I) or compound having general chemical Formula (I) wherein [¹⁷⁷Lu] is abent and suitable salts of inorganic or organic acids thereof, hydrates, complexes, esters, amides, and solvates thereof. Optionally the kit comprises a pharmaceutically acceptable carrier, diluent, excipient or adjuvant.

### Definitions

[¹⁷⁷Lu] is a radioisotope of Lutetium having a half-life of 6,7 days.

As used hereinafter in the description of the invention and in the claims, the terms “salts of inorganic or organic acids”, “inorganic acid” and “organic acid” refer to mineral acids, including, but not being limited to: acids such as carbonic, nitric, phosphoric, hydrochloric, perchloric or sulphuric acid or the acidic salts thereof such as potassium hydrogen sulphate, or to appropriate organic acids which include, but are not limited to: acids such as aliphatic, cycloaliphatic, aromatic, araliphatic, heterocyclic, carboxylic and sulphonic acids, examples of which are formic, acetic, trifluoracetic, propionic, succinic, glycolic, gluconic, lactic, malic, fumaric, pyruvic, benzoic, anthranilic, mesylic, fumaric, salicylic, phenylacetic, mandelic, embonic, methansulfonic, ethanesulfonic, benzenesulfonic, phantothenic, toluenesulfonic, trifluormethansulfonic and sulfanilic acid, respectively.

As used hereinafter in the description of the invention and in the claims, the terms “amino acid sequence” and “peptide” are defined herein as a polyamide obtainable by (poly)condensation of at least two amino acids.

As used hereinafter in the description of the invention and in the claims, the term “amino acid” means any molecule comprising at least one amino group and at least one carboxyl group, but which has no peptide bond within the molecule. In other words, an amino acid is a molecule that has a carboxylic acid functionality and an amine nitrogen having at least one free hydrogen, preferably in alpha position thereto, but no amide bond in the molecule structure. Thus, a dipeptide having a free amino group at the N-terminus and a free carboxyl group at the C-terminus is not to be considered as a single “amino acid” in the above definition. The amide bond between two adjacent amino acid residues which is obtained from such a condensation is defined as “peptide bond”. Optionally, the nitrogen atoms of the polyamide backbone (indicated as NH above) may be independently alkylated, e.g., with C₁-C₆-alkyl, preferably CH₃.

An amide bond as used herein means any covalent bond having the structure

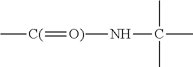

wherein the carbonyl group is provided by one molecule and the NH-group is provided by the other molecule to be joined. The amide bonds between two adjacent amino acid residues which are obtained from such a polycondensation are defined as “peptide bonds”. Optionally, the nitrogen atoms of the polyamide backbone (indicated as NH above) may be independently alkylated, e.g., with —C₁-C₆-alkyl, preferably —CH₃.

As used hereinafter in the description of the invention and in the claims, an amino acid residue is derived from the corresponding amino acid by forming a peptide bond with another amino acid.

As used hereinafter in the description of the invention and in the claims, an amino acid sequence may comprise naturally occurring and/or synthetic/artificial amino acid residues, proteinogenic and/or non-proteinogenic amino acid residues. The non-proteinogenic amino acid residues may be further classified as (a) homo analogues of proteinogenic amino acids, (b) β-homo analogues of proteinogenic amino acid residues and (c) further non-proteinogenic amino acid residues.

As used hereinafter in the description of the invention and in the claims, the term “peptide analogs”, by itself refers to synthetic or natural compounds which resemble naturally occurring peptides in structure and/or function.

All natural amino acids were represented by 3-letter codes. Unless otherwise stated all the aminoacids have L-configurations.

As used hereinafter in the description of the invention and in the claims, the term “statine analog” is defined as a di-peptidic mimetic with the following generic structure

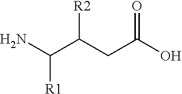

Statine R2═OH, R1 can be varied significantly but typically are the same as amino acid side chains
Statine Analogs R2═H, R1 can be varied significantly but typically are the same as amino acid side chains
Sta=Statine

The term “N-protecting group” (amine-protecting group) as employed herein by itself or as part of another group is known or obvious to someone skilled in the art, which is chosen from but not limited to a class of protecting groups namely carbamates, amides, imides. N-alkyl amines, N-aryl amines, imines, enamines, boranes, N—P protecting groups, N-sulfenyl, N-sulfonyl and N-silyl, and which is chosen from but not limited to those described in the textbook Greene and Wuts, Protecting groups in Organic Synthesis, third edition, page 494-653, which is hereby incorporated herein by reference.
Amino protecting groups are selected e.g. from the group comprising
Carbobenzyloxy (Cbz), tert-Butyloxycarbonyl (BOC) or 9-Fluorenylmethyloxycarbonyl (FMOC).

The term “O-protecting group” as employed herein refers to a carboxylic acid protecting group employed to block or protect the carboxylic acid functionality while the reactions involving other functional sites of the compound are carried out. Carboxy protecting groups are disclosed in Greene, “Protective Groups in Organic Synthesis” pp. 152-186 (1981), which is hereby incorporated herein by reference. Such carboxy protecting groups are well known to those skilled in the art, having been extensively used in the protection of carboxyl groups. Representative carboxy protecting groups are alkyl (e.g., methyl, ethyl or tertiary butyl and the like); arylalkyl, for example, phenethyl or benzyl and substituted derivatives thereof such as alkoxybenzyl or nitrobenzyl groups and the like.

Preferred O-protected compounds of the invention are compounds wherein the protected carboxy group is a lower alkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester.

O-protecting groups are selected e.g. from the group comprising
Methyl, Ethyl, Propyl, Butyl, t-Butyl or Benzyl.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The entire disclosure[s] of all applications, patents and publications, cited herein are incorporated by reference herein.

The following examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

Abbreviations

| | |
|---|---|
| MBHA | 4-Methylbenzhydrylamine |
| DIEA | N-ethyl-N-isopropylpropan-2-amine |
| | (diisopropylethylamine) |
| DBU | 1,8-Diazabicyclo(5.4.0)undec-7-en |
| HBTU | O-(benzotriazol-1-yl)-1,1,3,3- |
| | tetramethyluronium hexafluorophosphate |
| HOBt | 1-Hydroxybenzotriazole |
| TFA | Trifluoroacetic acid |
| RP-HPLC | Reversed phase HPLC |
| HPLC-MS | High performance liquid chromatography- |
| | mass spectrometry |
| DMA | N,N-Dimethylacetamide |
| DMF | N,N-Dimethylformamide |
| DMSO | Dimethylsulfoxide |
| EtOAc | Ethyl acetate |
| Fmoc | Fluorenylmethyloxycarbonyl |
| HPLC | High performance liquid chromatography |
| GBq | Giga Bequerel |
| MBq | Mega Bequerel |
| MS | Mass spectrometry |
| RT | Room temperature |
| ESI | Electrospray ionisation |
| PET | Positron Emission Tomography |
| GRPr | Gastrin-Releasing-Peptide receptor |
| PC-3 | Prostate cancer xenograft; androgen- |
| | independent |
| LNCaP | Prostate cancer xenograft; androgen- |
| | dependent |
| MIP | Maximum intensity projection |
| SPPS | solid phase peptide synthesis |
| MBHA | 4-methylbenzhydrylamine |
| TBCR | triazine based coupling reagent (herein |
| | used for 4-(4,6-dimethoxy-1,3,5-triazin-2- |
| | yl)-4-methylmorpholin-4-ium |
| | tetrafluoroborate) |

### 1. Experimental Chemistry

### Synthesis of DOTA-4-amino-1-carboxymethylpiperidine-D-Phe-Gln-Trp-Ala-Val-Gly-His-Sta-Leu-NH₂ a Non Radioactive Compound (1)

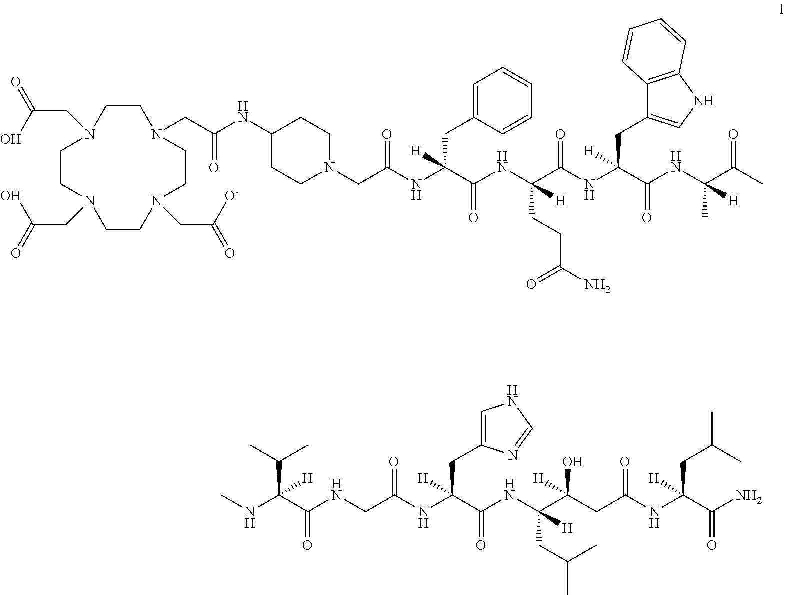

The peptide portion of the molecule H—R²—R³ (H is hydrogen) can be conveniently prepared according to generally established techniques known in the art of peptide synthesis, such as solid-phase peptide synthesis (SPPS). These methods are well documented in peptide literature. (Reference: “Fmoc Solid Phase Peptide Synthesis” A practical approach”, Edited by W. C. Chan and P. D. White, Oxford University Press 2000) (For Abbreviations see above). The publication cited herein is incorporated by reference herein.

Compound (1) was synthesized manually according to standard Fmoc chemistry, (Atherton E. Fluorenylmethoxycarbonyl-polyamide solid phase peptide synthesis. General principles and development, 1989) using Rink amide MBHA resin. The spacer and the chelator DOTA(¹Bu)₃ were consecutively coupled to the peptide with HATU as activating agent. The cleavage of peptides and simultaneous deprotection of the side chain protecting group was performed using TFA/H₂O/TIS (95/2.5/2.5). The peptide was purified by semi-preparative RP-HPLC and characterized by ESI-MS.

C₇₉H₁₁₈N₂₀O₁₉; calculated (m/z): 1639.9, found [M+K]⁺: 1678.1

### Synthesis of [¹⁷⁷Lu]-DOTA-4-amino-1-carboxymethylpiperidine-D-Phe-Gln-Trp-Ala-Val-Gly-His-Sta-Leu-NH₂ a Radioactive Compound (2)

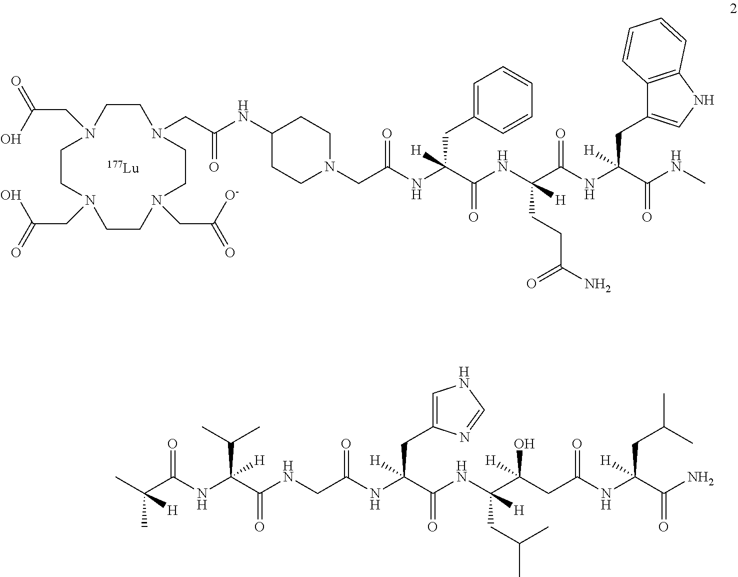

¹⁷⁷Lu-DOTA-peptide conjugates (2) were prepared by dissolving 10 μg of peptide in 250 μL of sodium acetate buffer (0.4 mol/L, pH 5.0) and by incubating with ¹⁷⁷LuCl₃ (110-220 MBq) for 30 min at 95° C. To obtain structurally characterized homogenous ligands, 1 equivalent of ^{nat}LuCl₃×5H₂O was added and the final solution incubated again at 95° C. for 30 min. For biodistribution and serum stability studies, the labeling was performed accordingly without the addition of cold metal. For injection, the radioligand solution was prepared by dilution with 0.9% NaCl (0.1% bovine serum albumin).

### Synthesis of [¹¹¹In]-DOTA-4-amino-1-carboxymethylpiperidine-D-Phe-Gln-Trp-Ala-Val-Gly-His-Sta-Leu-NH₂ a Radioactive Compound (3)

The synthesis of compound 3 is similar to compound 2 when using ¹¹¹InCl₃ and ^{nat}InCl₃.

### 2. Experimental Biological Data

### Example 1

### Binding Affinity to GRPr and Serum Stability Binding-Affinity Measurements

The binding-saturation experiments were performed using increasing concentrations of the compound 2 [^{177/nat}Lu] and compound 3 [^{111/nat}In] ranging from 0.1 to 1,000 nmol/L. For the blocking experiments 0.8 mmol/L of blocking agent was used. For each radioligand, triplicates were prepared for every concentration, for both total binding and nonspecific binding. Before adding the radioligands to the wells, the plates were placed on ice for 30 min. After adding the specific blocking agents and radioligands, the plates were incubated for 2 h at 4° C. After this time interval, the binding buffer was aspirated and the cells were washed twice with ice-cold phosphate-buffered saline (PBS, pH 7.4); this represented the free fraction. The cells were then collected with 1 N NaOH; this corresponded to the bound fraction. Specific binding was calculated by subtracting non specific binding from total binding at each concentration of radioligand.

The affinity (K_{d}) of the radioligand for the receptor and the binding site density (Bₘₐₓ) were calculated from Scatchard plots of the data using Origin 7.5 software (Microcal Software, Inc., Northampton, Mass.). In comparison with the In-111-labeled peptide (compound 3), ¹⁷⁷Lu-peptide (compound 2) shows an even slightly enhanced binding affinity of the peptide Seq 1.

### See FIG. 1 (A+B)

### Serum Stability

To 1 mL of freshly prepared human serum, previously equilibrated in a 5% CO₂ environment at 37° C., we added 0.03 nmol ¹⁷⁷Lu-labeled peptide ready to use solution (compound 2). The mixture was incubated in a 5% CO₂, 37° C. environment. At different time points, 100-μL aliquots (in triplicate) were removed and treated with 200 μL of EtOH to precipitate serum proteins. Samples were then centrifuged for 15 min at 500 rpm. 50 μL of supernatant were removed for activity counting in a micro-well counter, the sediment was washed twice with 1 mL of EtOH and counted, and the activity in the supernatant was compared with the activity in the pellet to give the percentage of peptides not bound to proteins or radiometal transferred to serum proteins. The supernatant was analyzed with HPLC (eluents: A=0.1% trifluoroacetic acid in water and B=acetonitrile; gradient: 0 min 95% of A; 20 minutes 50% of A) to determine the stability of the peptide in serum. In vitro compound 2 showed remarkable stability in human serum up to 4 days of incubation.

### See FIG. 2

### Example 2

### Biodistribution of Compound 2 in PC-3-Bearing Mice at Time 1 h, 4 h, 24 h, 48 h and 72 h

Biodistribution was investigated in NMRI nude mice bearing subcutaneous PC-3 tumors in the right hind limb at different time-points. Body weight of the male mice was approx. 30 g, 3 animals were investigated per time-point. After injecting an intravenous dose into the tail vein, mice were sacrificed at indicated time points and dissected organs were analyzed by radioactive counting. An administration dose of 100 μL was applied per animal with a mean activity of 86 kBq.

At indicated time points urine and feces were quantitatively collected. At the same time points, animals were sacrificed and exsanguinations under isoflurane anesthesia and the following organs and tissues were removed for measurement of [¹⁷⁷Lu] using the gamma-counter: spleen, liver, gallbladder, kidneys, lung, femur, heart, brain, fat, thyroid, muscle, skin, blood, tail, stomach (without content), prostate, intestine (with content), pancreas, adrenals, and the remaining body (designated as carcass).

Whole organs and tissues or aliquots were weighted and measured in a gamma-counter for radioactivity. In order to get the amount of total radioactivity administered in each animal (=100%) 3 aliquots (100 μL each) of the injected solution were always measured in parallel. The results of the biodistribution and excretion are reported as percent of injected dose per gram of tissue (% ID/g) and percent of injected dose per organ (% ID), respectively. All data are given as mean value±standard deviation, which were calculated by using all animals per time and sample.

TABLE 1
Biodistribution of compound 2 in PC-3-bearing mice

| | Timepoints: 1 h/4 h/24 h/48 h/72 h p.a | | | | | | | | | |
|---|---|---|---|---|---|---|---|---|---|---|
| | inject. volume: 100 μl i.v | | | | | | | | | |
| | inject. dose: 85.56 KBq (2.31 μCi) | | | | | | | | | |
| | Lu-177-DOTA-4-amino-1-carboxymethyl-piperidine-D-Phe-Gln-Trp-Ala-Val-Gly-His-Sta-Leu-NH2 | | | | | | | | | |
| | inoculation: PC-3 cells (human prostate cancer): s.c. inoculation of 2 × 10⁶ cells/100 μl Matrigel | | | | | | | | | |
| | species: Nude mice (NMRI nu/nu, male) | | | | | | | | | |
| Timepoint: | 1.0 h | | 4.0 h | | 24.0 h | | 48.0 h | | 72.0 h | |
| Weight (g): | | 29.46 | | 27.05 | | 26.61 | | 28.51 | | 27.62 |
| % ID/g | | S.D. | | S.D. | | S.D. | | S.D. | | S.D. |
| Spleen | 0.284 | 0.187 | 0.152 | 0.048 | 0.060 | 0.021 | 0.056 | 0.024 | — | — |
| Liver | 0.151 | 0.002 | 0.134 | 0.011 | 0.046 | 0.007 | 0.036 | 0.003 | 0.030 | 0.003 |
| Kidney | 1.715 | 0.448 | 1.709 | 0.285 | 0.568 | 0.211 | 0.285 | 0.069 | 0.175 | 0.019 |
| Lung | 0.364 | 0.069 | 0.083 | 0.017 | 0.019 | 0.005 | 0.048 | 0.029 | — | — |
| Bone | 0.096 | 0.009 | 0.052 | 0.013 | — | — | — | — | — | — |
| Heart | 0.143 | 0.033 | 0.037 | 0.015 | — | — | — | — | — | — |
| Brain | 0.028 | 0.003 | 0.014 | 0.006 | — | — | — | — | — | — |
| Fat | 0.511 | 0.320 | 0.115 | 0.050 | — | — | — | — | — | — |
| Thyroid | 0.185 | 0.087 | — | — | — | — | — | — | — | — |
| Gallbladder | 0.440 | 0.250 | 0.470 | 0.340 | — | — | — | — | — | — |
| Muscle | 0.072 | 0.009 | 0.020 | 0.010 | — | — | — | — | — | — |
| Tumor | 5.599 | 1.663 | 8.099 | 1.606 | 6.059 | 1.534 | 3.575 | 0.813 | 2.490 | 0.498 |
| Skin | 0.409 | 0.164 | 0.145 | 0.063 | 0.055 | 0.020 | 0.074 | 0.077 | 0.040 | 0.027 |
| Blood | 0.310 | 0.002 | 0.056 | 0.000 | 0.004 | 0.000 | 0.006 | 0.005 | — | — |
| Tail | 2.230 | 0.871 | 0.953 | 0.511 | 0.436 | 0.229 | 0.194 | 0.061 | 0.313 | 0.195 |
| Stomach | 2.562 | 1.100 | 1.361 | 0.270 | 0.081 | 0.021 | 0.042 | 0.016 | 0.020 | 0.004 |
| Prostate | 0.289 | 0.117 | — | — | — | — | — | — | — | — |
| Intestine | 1.632 | 0.218 | 1.163 | 0.225 | 0.137 | 0.084 | 0.110 | 0.085 | 0.084 | 0.061 |
| Pancreas | 18.714 | 0.979 | 5.166 | 0.785 | 0.579 | 0.080 | 0.288 | 0.113 | 0.206 | 0.008 |
| Adrenals | 3.038 | 1.628 | 2.190 | 0.291 | 1.528 | 0.238 | 1.064 | 0.745 | 1.031 | 0.295 |
| Time point: | 1.0 h | | 4.0 h | | 24.0 h | | 48.0 h | | 72.0 h | |
| % ID | | S.D. | | S.D. | | S.D. | | S.D. | | S.D. |
| Spleen | 0.034 | 0.029 | 0.015 | 0.010 | 0.005 | 0.001 | 0.003 | 0.001 | — | — |
| Liver | 0.302 | 0.018 | 0.175 | 0.007 | 0.060 | 0.007 | 0.054 | 0.005 | 0.040 | 0.003 |
| Kidney | 0.990 | 0.347 | 0.691 | 0.134 | 0.267 | 0.070 | 0.152 | 0.032 | 0.092 | 0.012 |
| Lung | 0.102 | 0.022 | 0.024 | 0.004 | 0.005 | 0.001 | 0.013 | 0.008 | — | — |
| Bone | 0.005 | 0.001 | 0.003 | 0.000 | — | — | — | — | — | — |
| Heart | 0.022 | 0.007 | 0.005 | 0.002 | — | — | — | — | — | — |
| Brain | 0.011 | 0.001 | 0.005 | 0.002 | — | — | — | — | — | — |
| Fat | 0.018 | 0.001 | 0.006 | 0.006 | — | — | — | — | — | — |
| Thyroid | 0.006 | 0.001 | — | — | — | — | — | — | — | — |
| Gallbladder | 0.000 | 0.005 | 0.000 | 0.010 | — | — | — | — | — | — |
| Muscle | 0.014 | 0.003 | 0.003 | 0.001 | — | — | — | — | — | — |
| Tumor | 1.119 | 0.807 | 3.072 | 0.637 | 2.191 | 1.117 | 1.051 | 0.440 | 0.550 | 0.121 |
| Skin | 1.806 | 0.839 | 0.515 | 0.202 | 0.162 | 0.073 | 0.246 | 0.239 | 0.152 | 0.110 |
| Blood | 0.797 | 0.017 | 0.119 | 0.014 | 0.007 | 0.001 | 0.013 | 0.011 | — | — |
| Tail | 1.836 | 0.833 | 0.694 | 0.408 | 0.337 | 0.181 | 0.150 | 0.051 | 0.263 | 0.175 |
| Stomach | 0.614 | 0.223 | 0.321 | 0.053 | 0.018 | 0.004 | 0.010 | 0.004 | 0.005 | 0.001 |
| Prostate | 0.003 | 0.001 | — | — | — | — | — | — | — | — |
| Intestine | 4.792 | 0.476 | 2.318 | 0.168 | 0.385 | 0.236 | 0.317 | 0.248 | 0.211 | 0.152 |
| Pancreas | 8.025 | 0.288 | 1.735 | 0.389 | 0.196 | 0.017 | 0.076 | 0.038 | 0.046 | 0.009 |
| Adrenals | 0.014 | 0.009 | 0.013 | 0.002 | 0.007 | 0.002 | 0.007 | 0.004 | 0.005 | 0.001 |
| Summary | | S.D. | | S.D. | | S.D. | | S.D. | | S.D. |
| Recovery | 96.950 | 4.038 | 92.830 | 1.904 | 86.280 | 16.138 | 91.600 | 6.344 | 91.750 | 12.838 |
| Organs | 20.080 | 0.382 | 9.640 | 0.888 | 3.640 | 0.741 | 2.090 | 0.366 | 1.370 | 0.478 |
| Carcass | 5.030 | 0.099 | 1.480 | 0.759 | 0.370 | 0.162 | 0.290 | 0.214 | 0.210 | 0.057 |
| Urine | 71.820 | 11.462 | 63.130 | 16.792 | 76.300 | 17.411 | 74.050 | 18.707 | 63.690 | 43.536 |
| Faeces | — | — | 18.570 | 19.267 | 5.980 | 1.712 | 15.210 | 12.426 | 26.490 | 31.234 |

Tumor/Tissue ratios:

| | | S.D. | | S.D. | | S.D. | | S.D. | | S.D. |
|---|---|---|---|---|---|---|---|---|---|---|
| T/spleen | 27.64 | 24.07 | 60.41 | 34.17 | 115.53 | 60.07 | 68.34 | 15.84 | — | — |
| T/liver | 37.09 | 10.59 | 60.01 | 7.01 | 131.75 | 21.28 | 100.56 | 21.36 | 84.93 | 22.61 |
| T/kidney | 3.25 | 0.12 | 4.83 | 1.19 | 11.93 | 6.31 | 12.83 | 2.68 | 14.38 | 3.75 |
| T/lung | 15.23 | 1.68 | 100.73 | 28.92 | 352.79 | 158.26 | 85.54 | 28.06 | — | — |
| T/bone | 57.84 | 11.71 | 156.36 | 10.32 | — | — | — | — | — | — |
| T/heart | 41.52 | 21.16 | 241.71 | 92.80 | — | — | — | — | — | — |
| T/brain | 201.41 | 39.71 | 670.63 | 288.07 | — | — | — | — | — | — |
| T/fat | 14.88 | 12.57 | 81.45 | 43.48 | — | — | — | — | — | — |
| T/muscle | 80.43 | 33.64 | 459.24 | 149.43 | — | — | — | — | — | — |
| T/skin | 15.76 | 10.36 | 59.98 | 15.56 | 122.45 | 67.66 | 79.48 | 44.75 | 83.73 | 49.57 |
| T/blood | 18.07 | 5.46 | 143.89 | 28.98 | 1718.99 | 405.73 | 878.55 | 567.14 | 1564.46 | 1129.51 |
| T/stomach | 2.25 | 0.32 | 6.17 | 2.01 | 77.62 | 29.83 | 91.71 | 27.24 | 128.40 | 40.29 |
| T/intestine | 3.53 | 1.49 | 7.01 | 0.90 | 56.82 | 39.92 | 47.21 | 33.24 | 43.54 | 30.43 |
| T/thyroid | 31.67 | 5.95 | — | — | — | — | — | — | 74.41 | 78.32 |
| T/prostate | 22.41 | 14.85 | — | — | — | — | — | — | — | — |
| T/ | 0.30 | 0.07 | 1.63 | 0.61 | 10.82 | 3.94 | 13.17 | 3.57 | 12.03 | 2.03 |
| pancreas | | | | | | | | | | |
| T/adrenals | 2.32 | 1.79 | 3.73 | 0.78 | 4.12 | 1.58 | 4.05 | 1.48 | 2.60 | 1.01 |

*Tissue Aliquots only

### Example 3

### Dosimetry

The biodistribution data of compound 2 in PC-3-tumor bearing mice (see example 2) were used for dosimetry calculations by the MIRD (Medical Internal Radiation Dosimetry) methodology to estimate mouse-organ self-to-self doses. Time activity (kinetic) data were modeled to produce the residence times for compound 2.

Dosimetry calculated by the Medical Internal Radioation Dose (MIRD) methodology showed an excellent therapeutic window in mice (regarding kidneys and pancreas). Doses of 150-200 Gy in the tumor could be achieved considering a maximum activity of 450 MBq to be injected per animal. Kidneys were not critical instead it was the pancreas to be the dose limiting organ. (In contrast to rodent pancreas, human pancreas expresses only very low amounts of the GRPr.)

### See FIG. 3

### Example 4

### Comparison Compound 2 with ¹⁷⁷Lu-AMBA

Biodistributions in PC-3 tumor bearing mice show the advantages of the bombesin antagonist compound 2 (example 2, table 1) comparing to the published radiotherapeutic bombesin agonist ¹⁷⁷Lu-AMBA from Bracco in terms of tumor retention over time and tumor/kidney-ratio.

TABLE 2
Comparison of biodistribution of the two compound 2 in PC-3-tumor
bearing mice 1 h p.i. and 24 h p.h. expressed as % ID/g (n = 3).

| | Lu-177-AMBA* | | compound 2 | |
|---|---|---|---|---|
| % ID/g | 1 h p.i. | 24 h p.i. | 1 h p.i. | 24 h p.i. |
| Blood | 0.25 (±0.13) | 0.02 (±0.01) | 0.31 | 0.004 |
| Tumor | 5.03 (±1.44) | 3.40 (±0.95) | 5.60 (±1.66) | 6.06 (±1.53) |
| Liver | 0.22 (±0.11) | 0.39 (±0.57) | 0.15 | 0.05 (±0.01) |
| Kidneys | 7.61 (±2.87) | 2.69 (±0.63) | 1.72 (±0.45) | 0.57 (±0.21) |

*Pangione S, Nunn A D, Q J Nucl Med Mol Im 2006; 50: 310-21

### Example 5

### Radionuclide Therapy Study with Repeated Injections

A first therapy study was conducted on 25 nude mice (15-20 g) subcutaneously implanted with PC-3 (10⁶ million of cells). For toxicity study the same therapy protocol was applied to 25 CD1 mice. Thirteen days after implantation the mice were randomly divided in 5 groups and treated as described below:
1. 100 pmol/6 MBq of compound 2
2. 200 pmol/12 MBq of compound 2
3. 400 pmol/24 MBq of compound 2
4. 200 pmol of ^{nat}compound 2
5. PBS

According to the protocol agreed upon, three injections per week (days 0, 2, 4) were done and the procedure was repeated (days 14, 16, 18) after one pause week. Based on the biodistribution data of compound 3 with ¹¹¹In we observed that injections after 48 h would maintain stable the uptake in the tumor. The pause week is, in our opinion, important for the safety of the animals. The mice were periodically monitored by measuring tumor size and body mass. Animals with loss of >20% of their original weight or with tumor size >20 mm in diameter were sacrificed. Tumor sizes were determined by caliper measurements in two dimensions and tumor volumes were calculated assuming an elliptical shape. Tumor, kidneys and pancreas were prepared for histological investigation (where possible).The animals treated with the higher doses showed reduction of the tumor mass and in many cases complete remission.

The animals treated with lower compound 2 radioactivity dose showed, mainly, an increasing of tumor volume except for the mouse No. 5. These animals, in fact, had a small tumor volume when the therapy started and a complete remission was observed.

The animals belonging to the second and third groups showed a good response to the treatment. Complete remission was observed for almost all the animals. Fifty days after the treatment was initiated few animals (2 of the second and 1 of the third group) showed fast regrowing of the tumor and they were sacrificed. Fast tumor growth was observed for the mice belonging to the forth and fifth groups. On day 26 the animal No. 1 of the first group and the animal No. 4 of the fifth group were treated with a single dose injection (400 pmol/50 MBq) in order to study the effect of high radioactivity dose on an advanced tumor. The tumor volume decreased rapidly till a complete remission in case of the mouse No. 4 while a recurrence of the tumor was observed for the mouse No. 1.

All CD1 animals are alive and look healthy; the body weight increased and it stabilized after 5-6 months.

At the end of the study no compound (radio-peptide) related histopathological changes were observed in the treated mice submitted for histological examination when compared to controls.

### See FIGS. 4-8.

### Example 6

### Radionuclide Therapy Study with Single Injection

Study design was similar as in example 5 with following modifications:
Inoculation of 5×10⁶ PC-3 tumor cells,
Single injection of 37 MBq compound 2 on day four,
2 repeated studies with a set of 5 mouses.

In two subsequent studies the effect on PC-3 tumor growth by treatment with compound 2 is confirmed.

See FIGS. 9-10.

1. A conjugate of Formula I
[¹⁷⁷Lu]—R¹—R²—R³ (I)
wherein
R¹ is a metal chelator suitable for chelating [¹⁷⁷Lu],
R² is a spacer linked to N-terminal of R³ or a covalent bond,
R³ is a bombesin analog peptide antagonist of sequence from seq 1 to 4
Seq 1: D-Phe-Gln-Trp-Ala-Val-Gly-His-Sta-Leu-NH₂;
Seq 2: D-Phe-Gln-Trp-Ala-Val-Gly-His-Leuψ(CHOH—CH₂)—(CH₂)₂—CH₃;
Seq 3: D-Phe-Gln-Trp-Ala-Val-Gly-His-Leuψ(CH₂NH)-Phe-NH₂; and
Seq 4: D-Phe-Gln-Trp-Ala-Val-Gly-His-Leuψ(CH₂NH)-Cys-NH₂,
or a and pharmaceutically acceptable salt thereof.

2. The conjugate according to claim 1 wherein R¹ is a DOTA-, NODASA-, NODAGA-, NOTA-, DTPA-, EDTA-, TETA-, or TRITA-based chelator of close analog thereof.

3. The conjugate according to claim 1 that is [¹⁷⁷Lu]-DOTA-4-amino-1-carboxymethylpiperidine-D-Phe-Gln-Trp-Ala-Val-Gly-His-Sta-Leu-NH₂.

4. A composition comprising a compound of Formula I according to claim 1, and a pharmaceutically acceptable carrier or diluent.

5. A conjugate of formula I according to claim 1 or a pharmaceutical composition thereof for radiotherapy of cancer.

6. A method for radiotherapy comprising administering to a subject in need thereof compound of formula I according to claim 1, or composition thereof, in a therapeutically effective amount, and after localization of said compound of formula I or composition thereof in the desired tissues, subjecting the tissues to irradiation to achieve the desired therapeutic effect.

7. A method according to claim 6 wherein the desired tissue is a tumor and/or metastases located in or originated from prostate, lung or breast.

8. A method for obtaining a bombesin analog peptide antagonist conjugate of formula I
[¹⁷⁷Lu]—R¹—R²—R³ (I)
wherein
R¹ is a metal chelator suitable for chelating [¹⁷⁷Lu],
R² is a spacer linked to N-terminal of R³ or a covalent bond,
R³ is a bombesin analog peptide antagonist of sequence from seq 1 to 4
Seq 1: D-Phe-Gln-Trp-Ala-Val-Gly-His-Sta-Leu-NH₂;
Seq 2: D-Phe-Gln-Trp-Ala-Val-Gly-His-Leuψ(CHOH—CH₂)—(CH₂)₂—CH₃;
Seq 3: D-Phe-Gln-Trp-Ala-Val-Gly-His-Leuψ(CH₂NH)-Phe-NH₂; and
Seq 4: D-Phe-Gln-Trp-Ala-Val-Gly-His-Leuψ(CH₂NH)-Cys-NH₂;
said method comprising:
Optionally coupling a spacer R² to a bombesin analog peptide antagonist R³ for obtaining R²—R³,
Coupling R²—R³ to a suitable chelator, and
Radiochelating the bombesin analog peptide antagonist conjugate R¹—R²—R³ with [¹⁷⁷Lu].

9. A kit comprising a sealed vial containing a predetermined quantity of a compound having general chemical Formula (I) according to claim 1, or a suitable salt of an inorganic or organic acid thereof, or a hydrate, complex, ester, amide, or solvate thereof.
